(19) United States
(12) Patent Application Publication
(10) Pub. No.: US 2009/0060780 A1
(43) Pub. Date: Mar. 5, 2009

(54) Device and Method for the Treatment and/or Decontamination of Surfaces

(75) Inventors: Konstantin Walter, Hirschberg (DE); Johannes Scharrer, Mannheim (DE)

Correspondence Address: LERNER GREENBERG STEMER LLP, P O BOX 2480, HOLLYWOOD, FL, 33022-2480, US

(73) Assignee: Westinghouse Electric Germany GmbH, Mannheim (DE)

(21) Appl. No.: 12/202,945

(22) Filed: Sep. 2, 2008

(30) Foreign Application Priority Data
Aug. 31, 2007 (DE) 10 2007 041 408.2

(51) Int. Cl. A61L 2/025; A61L 2/08; A61L 2/12

(52) U.S. Cl. 422/20; 422/186; 422/22; 422/128; 422/21

(57) ABSTRACT

A device for the treatment and/or decontamination of surfaces has at least one generator configuration with which directed waves, in particular of an electromagnetic type, can be generated and its energy transmitted by a transmission device onto contaminated surface deposits, in particular surface deposits of a reactor pressure vessel and/or reactor internals. The surface deposits are dissolved and/or sublimated by the directed waves. In a corresponding method, the device is used to generate the directed waves and bring about transmissions of energy onto the contaminated surface deposits, in particular surface deposits of a reactor pressure vessel and/or reactor internals, in such a way that these surface deposits are dissolved and/or sublimated.

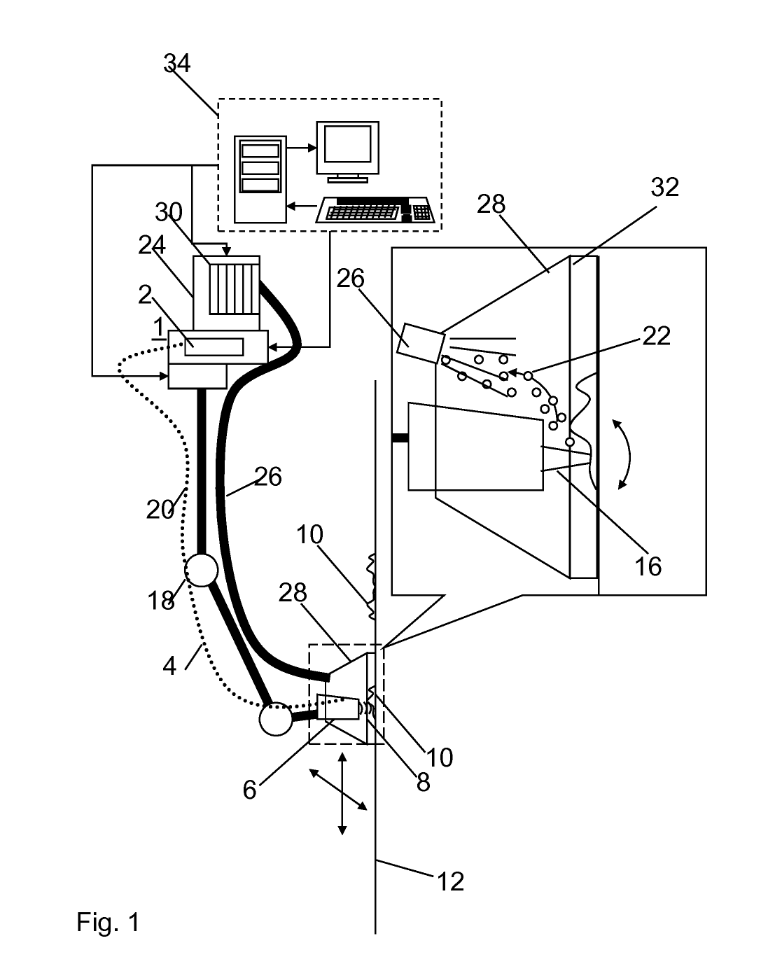

### CROSS-REFERENCE TO RELATED APPLICATION

This application claims the priority, under 35 U.S.C. §119, of German application DE 10 2007 041 408.2, filed Aug. 31, 2007; the prior application is herewith incorporated by reference in its entirety.

### BACKGROUND OF THE INVENTION

1. Field of the Invention

The invention relates to a device and a method for the treatment and/or decontamination of surfaces, in particular surfaces of reactor internals, such as for example a reactor pressure vessel. The method does not require the use of solvents and/or harsh chemicals.

Conventionally, loose and adhering radioactive contamination, in particular radioactive surface oxides, are removed by using mechanical and/or chemical treatment processes, such as for example brushing, wiping or polishing, in some cases with the aid of washing substances and/or solvents, such as organic or non-organic acids, alkaline solutions, solvents, ultrasound or high-pressure cleaning with steam or water.

However, mechanical processes are disadvantageously not very efficient because they can only be accomplished with considerable expenditure, in particular with respect to equipment, operating materials, residual materials requiring disposal and the treatment time that has to be spent. Conventionally, it is also not possible to proceed in a selective manner, since known methods do not differentiate between base material, such as for example the actual material of the reactor pressure vessel, and the contaminated oxide films deposited on the base material, and they attack and/or damage both materials equally during the decontamination or cleaning process. In addition, mechanical treatment tools generally leave traces of the treatment behind and, by their structural configuration alone, are spatially limited in their use. Simultaneously efficient and gentle decontamination of surfaces has not so far been possible.

### SUMMARY OF THE INVENTION

It is accordingly an object of the invention to provide a device and a method for the treatment and/or decontamination of surfaces which overcome the above-mentioned disadvantages of the prior art methods and devices of this general type, which provides an efficient and as gentle as possible way of decontaminating surfaces.

The device according to the invention accordingly contains at least one generator configuration and a device by which directed waves, in particular of an electromagnetic type, but also ultrasonic waves and/or x-rays and/or γ rays, can be generated and transmissions of energy onto contaminated surface deposits, in particular surface deposits of a reactor pressure vessel and/or of reactor internals, can be brought about and/or these surface deposits, in particular contaminated oxide films, can be dissolved and/or sublimated.

In the case of the method according to the invention for the treatment and/or decontamination of surfaces, which likewise achieves the set object, a corresponding device is used for generating directed waves, in particular of an electromagnetic type, but also ultrasonic waves and/or x-rays and/or γ rays, and for bringing about transmissions of energy onto contaminated surface deposits, in particular surface deposits of a reactor pressure vessel and/or of reactor internals, in such a way that the surface deposits are dissolved and/or sublimated.

In an advantageous refinement, the generator configuration contains one or more components, at least one source and/or a waveguide or a beam guiding system and/or a coupling-out element being provided as components and/or the components being in operative connection with one another.

In a preferred configuration, the generator configuration accordingly has at least one laser as a source and/or at least one optical system, in particular with a lens and/or diaphragm and/or focusing element, as the coupling-out element and/or at least one optical fiber as the waveguide or beam guiding system, the laser and the coupling-out element interacting and/or being connected by the at least one optical fiber. The laser used may in this case be formed in particular as a solid-state laser or a pulsed solid-state laser, for example as a titanium:sapphire laser or else an Nd:YAG/YLF laser, and thereby contain wavelengths in the range of about 266-1064 nm and/or pulse lengths of about 10 ns to 100 ns, or else as a CO₂ laser with wavelengths in the μm range, in particular about 10.6 μm. In the case of such lasers, pulse energies in the range of a few mJ to several 100 mJ can be achieved.

By use of the generated laser beam, a predeterminable transmission of energy per unit of time, of maximum pulse energy per pulse duration, onto a defined surface region, and consequently onto contaminated surface deposits present in the surface region, is brought about and/or the respective surface deposits, in particular in the form of oxide films, are heated, dissolved and sublimated. The transmission of energy to different materials, such as for example the base material of the reactor pressure vessel and the contaminated oxide film deposited on it, is thereby also determined by the absorption behavior of the different materials with respect to the electromagnetic waves or radiation that is respectively used. Also to be taken into account as a further factor concerning the level of the transmission of energy is the beam focusing area or the cross-sectional area of the beam. So, on account of the different absorption behavior of the base material and the oxide film, it is possible, for example, for a focus to be chosen such that, although the oxide film is dissolved and sublimated, the base material is not damaged or attacked. In this way, even already existing cracks can be decontaminated and/or treated in this way, virtually without contact, without undergoing any further damage. Such treatment can even be carried out depth-selectively by suitable choice of the respective laser energy and/or pulse energy and/or focusing. So, it is even possible to perform surface treatment and/or decontamination layer by layer in a number of working steps, which allows particularly gentle handling of the base material, for example of the reactor pressure vessel and/or its internals.

It is also possible as a development to provide at least one scanning device, with which a predeterminable region of the surface area can be homogeneously scanned with electromagnetic waves. The size and/or geometry of the respective surface area region and the scanning rate and/or resolution can in this case be prescribed.

A larger surface can in this case be treated or decontaminated by arranging a number of such surface area regions in series. The number of surface area regions and their arrangement is in this case adapted to the shape and/or size of the surface to be treated or to be decontaminated.

Alternatively, smaller surface area regions and individual deposits can be selectively treated and/or dissolved and sublimated by fixing the respective scanning region.

Accordingly, in a further refinement, the scanning device contains at least one deflecting element, in particular a movable mirror or prism, for example for the controlled directing or deflecting of laser beams, or a field generator, such as in particular an electromagnet, a coil or a capacitor or a combination thereof.

As a development, it is advantageously possible to provide at least one controlling/regulating device, which interacts with the at least one generator configuration and/or the at least one scanning device in such a way that the energy of the respective electromagnetic wave, in particular the laser light, and/or its transmission of energy and/or its direction of propagation and/or its point of impingement and/or the size and/or geometry of the predeterminable surface area region can be controlled and/or regulated.

It is accordingly possible by use of the controlling/regulating device in interaction with the scanning device, and in particular the at least one deflecting element, in particular by controlling/regulating the movement of the at least one movable mirror or prism, the field strength of at least one variable magnetic field and/or a variable electric and/or electromagnetic field, to bring about scanning of the predeterminable surface area region. The scanning may in this case be carried out continuously or step by step.

The amount of energy that can be transmitted or provided by the electromagnetic wave can in this case be homogeneously distributed over the respectively predeterminable surface area region.

For simplified handling, in an advantageous refinement it is possible to provide at least one handling device, on which at least one generator configuration or components thereof, such as in particular one end of a waveguide and/or a coupling-out element, and/or at least one further device, such as in particular a scanning device, can be arranged and/or with which these can be guided and/or positioned in relation to the contaminated surface or the surface to be treated.

The handling device may in this case have a handle or holding grip for the manual guidance and/or positioning of at least one generator configuration or components thereof and/or at least one scanning device.

As an alternative to this or in addition to it, it is possible to provide at least one manipulator or a robot, in particular a multiaxial industrial robot, with which the guidance and/or positioning of the generator configuration and/or scanning device can be carried out in an automated manner.

In order as far as possible to avoid exposure of the surroundings to radiation-contaminated, radioactive solid matter or particles during the decontamination and/or treatment process, in an advantageous development it is possible to provide a suction removal device for removing the dissolved and/or sublimated deposits by suction.

In this case, the suction removal device may also have a flexible suction hose and/or nozzle, it being possible for the respective nozzle to be arranged at the distal end of the respective hose.

The nozzle can advantageously be formed as a flat nozzle and/or can be adapted to the scanning region of the respective scanning device, in particular in such a way that the respective scanning region is covered as completely as possible in terms of surface area.

In a further refinement, the flat nozzle has a sealing device, which close off or seal the scanning region from the surroundings as completely as possible during the decontamination process, and consequently protect the ambient air to the greatest extent from being contaminated with contaminated particles.

In a further configurational variant, the nozzle has a clearance for leading through and/or receiving for coupling in at least one component of a generator arrangement, in particular a beam directing system and/or waveguide, an optical system, a laser or a laser beam.

For improved monitoring of the movement and/or position, it is advantageously possible to provide that the nozzle can be formed at least partly from transparent material, in particular a plastic.

It can also be provided that the suction removal device has at least one filter for the filtering and/or separating of dissolved, contaminated solid matter from the ambient air.

Alternatively or in addition to a laser, the generating configuration may also have at least one ultrasound source, an x-ray source, a gamma ray source (γ source) and/or a microwave generator.

Furthermore, the set object is also achieved by a method for the treatment and/or decontamination of surfaces, at least one of the aforementioned devices being used to generate directed electromagnetic waves and bring about transmissions of energy onto contaminated surface deposits, in particular surface deposits of a reactor pressure vessel and/or reactor internals, in such a way that they are dissolved and/or sublimated.

Laser beams which can be generated by a laser, in particular by a solid-state laser or a pulsed solid-state laser or an excimer laser (157 nm≦λ≦248 nm; ArF excimer laser λ=193 nm) can be advantageously used as electromagnetic sources. Furthermore, the generated laser beams can be focused by an optical system set up for this purpose, the laser beams being directed from the laser to the optical system by way of a waveguide or a beam guiding system, in particular an optical fiber or optical fiber bundle and/or a mirror or prism arrangement, and/or coupled out by way of the optical system.

Such laser beams generally are formed of electromagnetic waves with, for example, wavelengths in the range of about 157 nm to about 1060±4 nm, and/or a pulse length of about 4 ns to about 100 ns and/or a pulse energy of about 6 mJ to about 355 mJ, depending on the wavelength and type of laser used and/or focusing of the respective beam.

In terms of the method, it is accordingly possible by use of the generated laser beam for a predeterminable amount of energy to be transmitted per unit of time onto a defined surface region, and consequently onto contaminated surface deposits present in the surface region, and/or for contaminated deposits occurring in the respective surface region, in particular in the form of oxide films, to be heated, dissolved and/or sublimated.

In a configurational variant, a predeterminable region of the respective surface is homogeneously scanned with electromagnetic waves by at least one scanning device. In this case, the scanning of the respective surface area region is brought about by movement of at least one deflecting element, in particular a movable mirror or prism, or by field adaptation, in particular with respect to the field pattern and/or strength, of an electric and/or magnetic or electromagnetic field.

It is also possible to provide that the energy of the respective electromagnetic wave and/or of the transmission of energy brought about and/or its direction of propagation and/or its point of impingement and/or the size and shape of the scanned surface area region is controlled and/or regulated by a controlling/regulating device, in interaction with the generator configuration and/or scanning device.

In a further configuration, it is provided that movement, guidance, alignment and positioning of the generator configuration or components thereof and/or a scanning device in relation to the contaminated surface is carried out in a manual or automated manner by at least one handling device.

In the case of a further configurational variant, the generator configuration or components thereof and/or the scanning device as well as the respectively generated electromagnetic waves are guided and/or moved over the surface regions to be decontaminated at a predeterminable distance and/or in a predeterminable alignment by the at least one handling device.

In this case, the alignment, positioning and/or guidance may be carried out by at least one manipulator or a robot, in particular a multiaxial industrial robot.

In a development of the method, the dissolved and/or sublimated deposits are immediately sucked away by a suction removal device. The suction removal can in this case also be carried out by a flexible suction hose or a flexible suction line and a nozzle, the nozzle being arranged adjacent to the region of impingement of the electromagnetic waves and/or covering the latter.

A nozzle formed as a flat nozzle, which is adapted to the scanning region of the respective scanning device, in particular in such a way that the latter is covered in terms of surface area, can be used for the suction removal.

By way of developing the method, the air removed by suction is filtered, so that the dissolved and/or sublimated solid matter or deposits are separated in a filter, the filter being regularly cleaned and/or separated solid matter removed and properly disposed of or stored.

In an alternative configuration, waves in the form of ultrasonic waves, microwaves, x-rays, γ rays or a combination thereof are generated and/or, by the respectively generated waves, a predeterminable amount of energy is transmitted per unit of time onto a defined surface region, and consequently onto contaminated surface deposits present in the surface region, and/or contaminated deposits occurring in the respective surface region, in particular in the form of oxide films, are heated, dissolved and/or sublimated.

Accordingly, particularly homogeneous, efficient and gentle decontamination of surfaces, in particular of the reactor pressure vessel and/or the reactor internals, is made possible while avoiding mechanical treatment tools and the resultant traces of treatment and by using directed electromagnetic waves. It is also advantageously possible to dispense with the laborious provision and disposal (after use) of solvent and contaminated residues in comparatively large amounts.

A further advantage lies in the comparatively little expenditure and/or few, compact peripherals required for carrying out the method.

The further description of the invention, advantageous refinements and developments is based on a FIGURE and the associated exemplary embodiments.

Other features which are considered as characteristic for the invention are set forth in the appended claims.

Although the invention is illustrated and described herein as embodied in a device and a method for the treatment and/or decontamination of surfaces, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWING

The single FIGURE of the drawing is a diagrammatic illustration of a device for treating and/or decontaminating surfaces according to the invention.

### DETAILED DESCRIPTION OF THE INVENTION

Turning now to the single FIGURE of the drawing in detail, there is shown a device for the treatment and/or decontamination of surfaces. The device has by way of example, at least one generator configuration 1, containing a laser, for example a short-pulse excimer or solid-state laser 2, and a beam guiding system, for example an optical fiber bundle 4 if the solid-state laser 2 is used. The device further has an optical system with a focusing element (not explicitly shown), which is for example protected in a corresponding housing 6, being arranged, with which arrangement directed waves 8 can be generated and transmissions of energy onto contaminated surface deposits 10, in particular surface deposits of a reactor pressure vessel and/or reactor internals 12, can be brought about and/or these surface deposits 10 can be dissolved and/or sublimated.

By laser pulses of suitable intensity and duration, the correspondingly irradiated deposits are thereby dissolved or vaporized and/or transformed into a plasma.

Also provided is a scanning device, which can likewise be integrated in the housing 6 or be the housing and with which a predeterminable surface area region can be homogeneously scanned with, in particular, electromagnetic waves 8. For deflecting a respective laser beam 16, for example, a mirror or prism that can be moved in one or more axes and/or electromagnetic field generators and/or electromagnetic lenses is/are provided (not explicitly represented in the FIGURE).

In addition, a handling device is provided in the form of a multiaxial robot 18, arranged on which are an optical system, one end of a glass fiber bundle 20 for the optical connection of the laser 2 and the optical system, as well as the scanning device, and with which these can be guided and/or positioned in relation to the contaminated surface in an automated manner. The movement of the laser beam by the handling device 18 over the surface can in this case be performed in a vertical direction and/or horizontal direction. In the case of this movement, it can also be advantageously provided that the optical system is regulated to a uniform average distance from the surface. The removal of deposits can also be carried out layer by layer and/or step by step, by passing repeatedly over the corresponding structures and regions of the surface. In addition, a handle or holding grip can be provided for manual guidance and/or positioning. Alternatively, the handling device 18 may also be formed as a handheld device for exclusively manual operation and guidance, which appears to be appropriate in particular in cases of just local work and only small, almost punctiform surface area regions.

To also allow such handheld devices to be used in an automated manner, corresponding interfaces for attachment to a manipulator or robot 18 may be provided.

The constituents 22 of the previous deposits 10 that are dissolved and/or sublimated by laser beams 16 are immediately sucked away by a suction removal device 24. For better handling, the suction removal is performed by a flexible suction hose 26 or a flexible suction line and a nozzle 28 arranged on the end thereof. In the example shown here, the nozzle 28 is formed as a funnel-shaped flat nozzle 28, which is adapted to the surface area or scanning region that is passed over by the scanning device and completely covers it. Alternatively, the nozzle 28 used may also be of any other desired geometries and forms and, for example, be arranged laterally adjacent to the laser beam 16 and/or to the region of impingement of the electromagnetic waves.

The flat nozzle 28 also contains a sealing device 32, for example sealing lips of soft rubber or bristles, which are arranged at the end of the nozzle 28, between the nozzle 28 and the surface, and enclose the surface region of the nozzle 28. During the decontamination process, the sealing device 32 closes off and/or seals the scanning region from the surroundings as completely as possible, in order that the ambient air is protected to the greatest extent from contamination by contaminated particles and the exposure is kept as low as possible.

As shown in the FIGURE, in the nozzle 28 there is at least one receptacle, in particular a clearance, for leading through and/or coupling in the optical waveguide or optical fiber bundle 20 as well as the optical system and/or the scanning device 6.

For improved monitoring of the movement and/or position, it is advantageously possible to provide that the nozzle 28 can be formed at least partly from transparent material, in particular a plastic, or that a transparent viewing window can be provided.

The air that is sucked away is filtered in a filter 30 provided for the purpose, so that the dissolved and/or sublimated solid matter 22 is separated in the filter 30 and/or the filter 30 is regularly cleaned and/or the separated solid matter 22 is removed and properly disposed of or stored.

Also provided is at least one controlling/regulating device 34, which interacts with the laser 2, the optical system and the scanning device 6 in such a way that it is possible to control and/or regulate the energy of the respective laser beams, in particular the pulse energy, and/or its respective transmission of energy, for example by corresponding focusing, and/or its direction of propagation and/or its point of impingement and/or the scanning area and/or the scanning rate.

The suction removal device 24 also advantageously interacts with the controlling/regulating device.

The use of the optical fiber bundle 20 and the suction hose 26 allow the actual laser 2 with the energy supply and the actual suction removal device 24 with the filter 30 to be arranged spatially away from the surface 12 to be treated or to be decontaminated.

This is of advantage in particular when the space available is restricted, increases the freedom of movement and facilitates handling, since it is then only necessary to move and/or guide the waveguide or beam guiding system, in particular a light guide or optical fiber 20, as well as the optical system and possibly the scanning device 6 and the suction hose 26 with nozzle 28.

1. A device for at least one of treating and decontaminating surfaces, the device comprising:
at least one generator configuration for generating directed waves of energy; and
transmission means for transmitting the direct waves from said generator configuration onto contaminated surface deposits for at least one of dissolving the contaminated surface deposits and sublimating the contaminated surface deposits.

2. The device according to claim 1, wherein said generator configuration has at least one component selected from the group consisting of a source, a waveguide, a beam guiding system, and a coupling-out element being in operative connection with one another.

3. The device according to claim 1, wherein:
said generator configuration has at least one component selected from the group consisting of a laser functioning as a source, an optical system, an optical system with a focusing element functioning as a coupling-out element, an optical fiber, and a mirror system; and
said laser and said coupling-out element one of interacting and being connected by one of said at least one optical fiber and said mirror system.

4. The device according to claim 3, wherein said laser is selected from the group consisting of solid-state lasers, excimer lasers and pulsed solid-state lasers.

5. The device according to claim 3, wherein said laser generating a laser beam for bringing about a predeterminable transmission of energy per unit of time onto a defined surface region, and consequently onto the contaminated surface deposits present in the defined surface region, and the contaminated surface deposits, including oxide films, are heated, dissolved and sublimated.

6. The device according to claim 1, wherein said generator configuration has at least one further component selected from the group consisting of ultrasound sources, microwave generators, x-ray sources, γ ray sources and a combination of these components.

7. The device according to claim 1, further comprising at least one scanning device with which a predeterminable region of a surface area can be homogeneously scanned with the directed waves being electromagnetic waves.

8. The device according to claim 7, wherein said at least one scanning device has at least one of a deflecting element and a field generator.

9. The device according to claim 8, further comprising at least one controlling/regulating device interacting with at least one of said at least one generator configuration and said at least one scanning device such that at least one of the energy of the directed waves, a transmission of the energy of the directed waves, a direction of propagation of the directed waves and a point of impingement of the directed waves is at least one of controlled and regulated.

10. The device according to claim 9, wherein by use of said controlling/regulating device in interaction with said at least one deflecting element, a field strength of at least one of at least one variable magnetic field and an electromagnetic field is controlled for scanning a predeterminable surface area region.

11. The device according to claim 7, further comprising at least one handling device, on which at least one of said generator configuration, components of said generator configuration, and said scanning device are disposed.

12. The device according to claim 11, wherein by means of said handling device at least one of said generator configuration, said components of said generator configuration and said scanning device can be one of guided and positioned in relation to a surface to be decontaminated.

13. The device according to claim 11, wherein said handling device has one of a handle and a holding grip for at least one of manual guidance and positioning of at least one of said generator configuration, said components of said generating configuration and said scanning device.

14. The device according to claim 11, wherein said handling device contains one of a manipulator, a robot, and a multiaxial industrial robot.

15. The device according to one of claim 11, wherein a guidance and positioning of at least one of said generator configuration, said components of said generator configuration, said scanning device and said optical system can be carried out in an automated manner.

16. The device according to claim 1, further comprising a suction removal device for removing at least one of dissolved contaminated deposits and separated contaminated deposits by suction.

17. The device according to claim 16, wherein said suction removal device has a flexible suction hose and a nozzle disposed at a distal end of said flexible suction hose.

18. The device according to claim 17, wherein said nozzle is at least one of formed as a flat nozzle and adapted to a scanning region of said scanning device such that said scanning region is covered in terms of surface area.

19. The device according to claim 18, wherein said flat nozzle has a sealing device for one of closing off and sealing the scanning region from surroundings during a decontamination process, and consequently protect ambient air to a greatest extent from contaminants.

20. The device according to claim 17, wherein said nozzle has a receptacle for coupling in of at least one of an optical waveguide, a beam guiding device, a laser and a laser beam.

21. The device according to claim 17, wherein said nozzle is formed at least partly from a transparent material.

22. The device according to claim 17, wherein said suction removal device has at least one filter for at least one of filtering and separating of dissolved, contaminated solid matter from ambient air.

23. The device according to claim 1, wherein the contaminated surface deposits are surface deposits of at least one of a reactor pressure vessel and reactor internals.

24. The device according to claim 9, wherein:
said deflecting element is selected from the group consisting of a movable mirror and a movable prism; and
said field generator is selected from the group consisting of electromagnets, coils, capacitors and a combination thereof.

25. The device according to claim 24, wherein said controlling/regulating device controls and regulates a movement of said movable mirror.

26. The device according to claim 11, wherein said generator configuration has a waveguide and a coupling-out element, and at least one end of said waveguide and said coupling-out element are supported by said handling device.

27. The device according to claim 21, wherein said transparent material is plastic.

28. A method for at least one of treating and decontaminating surfaces, which comprises the steps of:
providing a device having at least one generator configuration for generating directed waves of energy and a transmitting means for transmitting the directed waves from the generator configuration onto contaminated surface deposits for at least one of dissolving the contaminated surface deposits and sublimating the contaminated surface deposits.

29. The method according to claim 28, which further comprises providing the generator configuration with a laser and generating laser beams, as the directed waves, with the laser.

30. The method according to claim 29, which further comprises focusing the laser beams with an optical system.

31. The method according to claim 29, which further comprises directing laser light from the laser to the optical system by way of one of an optical fiber, an optical fiber bundle, and a beam guiding system.

32. The method according to claim 28, which further comprises generating electromagnetic waves in a wavelength range of 157 nm to 1060±4 nm as the directed waves.

33. The method according to claim 29, which further comprises using the laser beams to transmit a predeterminable amount of energy per unit of time onto a defined surface region, and consequently onto the contaminated surface deposits present in the defined surface region, including contaminated deposits in a form of oxide films, for heating, dissolving and/or sublimating the contaminated surface deposits.

34. The method according to claim 28, which further comprises providing at least one scanning device for homogeneously scanning a predeterminable region of a respective surface containing the contaminated surface deposits with the directed waves, being electromagnetic waves.

35. The method according to claim 34, which further comprises performing the scanning of the predeterminable region of the respective surface by one of moving at least one deflecting element and by field adaptation of at least one of an electric field, a magnetic field and an electromagnetic field.

36. The method according to claim 34, which further comprises controlling and/or regulating at least one of the energy of the directed waves being electromagnetic waves, a transmission of the energy brought about, a direction of propagation of the directed waves, a point of impingement of the directed waves, and a size and shape of a scanned surface area region with a controlling/regulating device, interacting with at least one of the generator configuration and the scanning device.

37. The method according to according claim 28, which further comprises carrying out at least one of a movement, a guidance, an alignment and a positioning of at least one of the generator configuration, components of the generator configuration, and a scanning device in relation to a contaminated surface in one of a manual and an automated manner by use of at least one handling device.

38. The method according to claim 37, which further comprises performing at least one of guiding and moving at least one of the generator configuration, the components of the generator configuration, the scanning device, and the directed waves over surface regions to be decontaminated at a predeterminable distance and/or in a predeterminable alignment by use of the at least one handling device.

39. The method according to claim 37, which further comprises carrying out at least one of the alignment, the positioning and the guidance by means of at least one of a manipulator, a robot, and a multiaxial industrial robot.

40. The method according to claim 28, which further comprises sucking away at least one of dissolved deposits and sublimated deposits by a suction removal device.

41. The method according to according to claim 40, which further comprises carrying out the suction removal with one of a flexible suction hose and a flexible suction line with a nozzle, the nozzle being one of disposed adjacent to the region of impingement by the directed waves being electromagnetic waves and covering the region of impingement.

42. The method according claim 41, which further comprises forming the nozzle as a flat nozzle adapted to a scanning region of a scanning device for use in suction removal.

43. The method according to claim 40, which further comprises filtering air removed during a suction process, so that at least one of dissolved solid matter, sublimated solid matter and deposits are separated in a filter.

44. The method according to claim 43, which further comprises:
regularly cleaning the filter;
regularly removing separated solid matter; and
performing one of properly disposing the separated solid matter and storing the separated solid matter.

45. The method according to claim 28, which further comprises generating the directed waves in a form of one of ultrasonic waves, microwaves, x-radiation and γ radiation and a predeterminable amount of energy of the directed waves is transmitted per unit of time onto a defined surface region, and consequently onto the contaminated surface deposits present in the defined surface region, including the contaminated surface deposits in a form of oxide films, for heating, dissolving and/or sublimating the contaminated surface deposits.

46. The method according to claim 28, which further comprises:
generating the directed waves as an electromagnetic wave; and
directing the energy of the directed waves onto the contaminated surface deposits selected from the group consisting of surface deposits of a reactor pressure vessel and reactor internals.

47. The method according to claim 29, which further comprises selecting the laser from the group consisting of a solid-state laser, an excimer laser and a pulsed solid-state laser.

48. The method according to claim 34, which further comprises:
selecting the deflecting element from the group consisting of a movable mirror and a movable prism; and
performing the field adaptation with respect to at least one of a field pattern and a field strength.

49. The method according claim 42, which further comprises forming the nozzle to cover the scanning region.
